(19) United States
(12) Patent Application Publication
(10) Pub. No.: US 2015/0112393 A1
(43) Pub. Date: Apr. 23, 2015

(54) LATERAL PLATE FOR SPINAL FUSION

(71) Applicant: Trinity Medical, Inc., Las Vegas, NV (US)

(72) Inventor: Jason E. Garber, Las Vegas, NV (US)

(21) Appl. No.: 14/521,687

(22) Filed: Oct. 23, 2014

Related U.S. Application Data
(60) Provisional application No. 61/894,567, filed on Oct. 23, 2013.

(51) Int. Cl. A61B 17/70; A61F 2/44

(52) U.S. Cl. CPC A61B 17/7059; A61F 2/4455; A61B 2017/564
USPC 606/279; 606/290

(57) ABSTRACT

The present invention provides an improved spinal fixation system for treating disease or damage to the spinal column, and methods of using the same. The novel spinal fixation system includes a low-profile lateral fixation plate for fixing adjacent vertebral bodies having specially textured surfaces for interfacing with implant screws, two or more implant screws having roughed surfaces for interfacing with the low-profile fixation plate, and a means for tightening the implant screws and the low-profile fixation plate together. The low profile lateral fixation plate facilitates a more efficient and less intrusive spinal fusion procedure because it can be inserted and implanted through the same incision and/or channel used to excise an intervertebral disc and/or bone tissue. The lateral plate fixation system is configured fix adjacent vertebrae in proper position, thereby facilitating fusion of the adjacent vertebrae at the proper spacing and angle.

This application claims priority to U.S. Provisional Patent Application No. 61/894,567, filed Oct. 23, 2013, pending, which is incorporated herein by this reference in its entirety.

### BACKGROUND OF THE INVENTION

1. Field of the Invention

The present invention relates to a fixation device for fixing the position of bones for the purpose of bone fusion procedures, and methods of using the device. More particularly, the present invention relates to a low profile fixation plate system for fusing vertebrae, and methods of using the same.

2. Description of the Related Art

Spinal conditions, including damage to the vertebral bodies and intervertebral discs, can be severely detrimental to a patient's health. These structural components of the spine can be damaged by injury, age-related wear, or disease (e.g., autoimmune disease). Sufferers of such conditions may experience severe back pain and neurological dysfunction, such as weakness and sensory deficits. If left untreated, patients may suffer chronic pain and loss of sensory and motor function. Surgical intervention may be necessary to stabilize the affected portion of the spine.

One method of repair is spinal fixation and fusion, in which damaged vertebral tissue and/or damaged intervertebral disc(s) may be excised and the targeted vertebrae may be joined together to stabilize the spinal column and induce healing of the vertebrae. Pedicle, lateral, and oblique mounting devices may be used to secure corrective spinal instrumentation (e.g., surgical rods) to vertebrae that have been selected for fusion. Fixation and fusion of vertebrae is frequently accomplished using rods and plates to secure bone grafts or implants between adjacent vertebral members. However, conventional lateral plates for spinal fixation are over-engineered and difficult to use during surgery. Additionally, a separate incision is typically necessary for the implantation of the plate, giving rise to increased morbidity from the additional incision and longer surgery times.

It is therefore desirable to provide a spinal fixation system for stabilizing adjacent vertebrae for vertebral fusion using a low profile lateral fixation plate, and that eliminates the disadvantages of conventional fixation plates.

### SUMMARY OF THE INVENTION

The present invention provides an improved spinal fixation system for stabilizing vertebrae, properly aligning the spinal column, and/or relieving pressure on the spinal cord and other nerve tissue, and methods of using the same. The system includes a novel low-profile lateral fixation plate that can be introduced into relatively narrow incisions and channels, such as a channel for accessing and excising an intervertebral disc. The low-profile lateral fixation plate and other components of the spinal fixation system may be efficiently surgically implanted to correct conditions of the spine (e.g., degenerative intervertebral discs, damaged vertebral bodies, etc.), promote fusion of vertebrae, and/or relieve mechanical stresses imparted to the spinal cord that can result from conditions of the spine, without the need for a second incision for placement of a fixation plate.

The lateral fixation plate of the present invention may be configured for attachment to any of the vertebrae. In some implementations, and without limitation, the lateral fixation plate may be particularly suited for the thoracolumbar vertebrae. The lateral fixation plate may have a low profile and a shaped designed for attachment to adjacent vertebrae in order to fix the relative position of the vertebrae and facilitate fusion of the adjacent vertebrae. The low profile features may, for example, include a reduced thickness compared to conventional spinal fixation plates. The lateral fixation plate of the present invention may also include a reduced perimeter profile, to minimize its size and facilitate insertion through an incision without sacrificing the structural strength and integrity of the plate (e.g., the lateral fixation plate may have a “dumbbell” perimeter profile having a reduced width between the ends of the plate). To maintain the structural strength and integrity of the low-profile lateral fixation plate, the spinal fixation plate may vary in cross-section thickness to provide a minimized thickness at the perimeter, while maintaining structural strength and stability with a somewhat thicker central portion. The smaller perimeter thickness may provide for easier insertion of the low-profile fixation plate into lateral incisions, posteriolateral incisions, posterior incisions, etc., allowing the avoidance of an additional incision to insert the low-profile fixation plate. In some implementations, and without limitation, the thickness may smoothly taper from the central portion of the plate to the perimeter thereof, providing a smooth transition of thickness and no hard edges.

The low-profile of the lateral fixation plate may allow it to be inserted and implanted through various types of incisions. For example, the lateral fixation plate of the present invention can be implanted through incisions used in anterolateral and retroperitoneal approaches for interbody spine fusions. The lateral fixation plate system may be designed to perform a similar fixation function as pedicle screws without the need for an additional incision or the need to reposition the patient. This single approach fixation saves the patient from the morbidity of an additional incision and approach to the spine for adjunctive fixation. Additionally, the use of the lateral fixation plate of the present invention may save the surgeon and hospital significant time and money that would otherwise be spent applying posterior fixation procedures that require several additional steps.

In some embodiments, and without limitation, the lateral fixation plate may be paired with specially designed implant screws that create significant purchase or bite between the implant screws and receiving sockets or areas in the fixation plate. For example, and without limitation, the implant screws may have a collar or some other protrusion for engaging with the receiving sockets or areas of the engaging plate, and the protrusion may have a high-friction surface. A high-friction surface may include a textured surface, such as a surface that has been roughened, scribed, scored, hatched, stippled, etc. In further examples, and without limitations, the receiving sockets or areas of the fixation plate may have high-friction surfaces. In still further examples, and without limitation, both the collar and protrusion of the implant screws and the receiving sockets or areas of the fixation plate may both have high-friction surfaces.

In some implementations, and without limitation, the collar or protrusion of the implant screws and the receiving sockets or areas in the fixation plate may have complementary shapes that increase interfacing surface areas between them. For example, the implant screws may have collar or protrusion having a convex or protruding interfacing surface having a particular shape (e.g., a spherical cap, an ellipsoidal cap, a polygonal prism, a prolate spheroid cap, an oblate spheroid cap, conical, a rounded cone, etc.) and the fixation plate may have a receiving socket that has a concave or recessed interfacing shape that is complementary or at least partially complementary to the convex or protruding interfacing surface of the collar or protrusion. In other examples, and without limitation, the implant screws may have a collar or protrusion that has a concave or recessed interfacing surface (optionally, having a high-friction texture) and the fixation plate may have convex or protruding receiving area (optionally having a high-friction texture).

In embodiments that include high-friction surfaces on the implant screws and/or the fixation plate, the high-friction surface(s) may undergo cold welding when the interfacing surfaces of the implant screws and the fixation plate are in contact and significant pressure is applied to that interface (e.g., they are squeezed together by a tightening nut or other fastener over the lateral fixation plate). Cold welding is welding by pressure and without the supply of any substantial amount of external heat. In some implementations, and without limitation, the surfaces of the parts to be welded may be cleaned, such as by scouring or scratch-brushing, to produce metallic surfaces that are uncontaminated by matter foreign to the metal, and may then be placed in face-to-face contact, whereupon pressure may be applied to the contacting surfaces to cause the metal to cold flow into intimate and integrated contact, thereby producing a solid phase cold weld bond between the contacting surfaces. Easily deformable metals such as aluminum and copper can be cold-welded, but the process can be also achieved in medically acceptable metals, including, but not limited to titanium, titanium alloys (e.g., alloys of Ti, Al, and/or Nb), and stainless steel. Cold welding can also occur between the surfaces of two or more different metals (titanium alloy-stainless steel, etc.).

In some embodiments, and without limitation, the low-profile lateral fixation plate and/or the implant screws may include a surgically acceptable metal capable of undergoing cold welding when pressure is applied thereto. The specially textured metal surfaces of the implant screws and the lateral fixation plate may facilitate cold welding between the specially textured surfaces of the implant screws and the lateral fixation plate when the lateral fixation plate is tightened into position. The cold welding of the implant screws and the lateral fixation plate provides stable connections between the implant screws and the lateral fixation plate without slippage between the lateral fixation plate and the screws. The stable and reliable connection between the implant screws and the lateral fixation plate may allow the lateral fixation plate to stably hold the targeted vertebrae in the position for fusion without changes in the position of the plate or the positioning between the adjacent vertebrae joined by the lateral fixation plate. The stability provided by the cold welding between the lateral fixation plate and the implant screws may also allow for effective fixation and fusion using fewer implant screws (e.g., two screws rather than four or more, as is typical of conventional fixation systems). Further benefits and advantages will be apparent from the description of the embodiments of the invention.

In one aspect, the present invention relates to a spinal fixation system, including a low-profile fixation plate for stabilizing adjacent vertebrae having at least one textured surface and a hole running through the at least one textured surface, at least one screw having a second textured surface configured to interface with the at least one textured surface of the low-profile plate and a head configured to be placed through the hole, and at least one fastening member configured to tighten the screw to the low-profile fixation plate. In some embodiments, and without limitation, the spinal fixation system may additionally include a plate holding device that may be configured to (1) insert the low-profile plate into an incision and/or (2) to function as an anti-torque tool during the process of tightening the fastening member over the screw. The hole(s) running through the at least one textured surface may have a greater diameter(s) than the head(s) of the at least one screw, in order to allow the low-profile fixation plate to receive the head(s) when they are at various angles that may result from the particular position of the screw in the bone tissue. In some implementations, and without limitation, the spinal fixation system may include two or more screws and the low-profile fixation plate may include two or more textured surfaces, each having a hole for receiving a head of one of the screws. In some implementations, and without limitation, the spinal fixation system may be limited to two screws, which may reduce the time required for implanting the spinal fixation system and may reduce possible damage, morbidity, and discomfort that may result from the implantation of additional screws.

In another aspect, the present invention relates to a spinal fixation plate, including a low profile design, at least two holes for coupling with surgical screws, a bottom side having at least two concavities, each of the concavities surrounding one of the at least two holes, textured surfaces within the at least two concavities, and a recess for receiving an anti-torque tool. In some implementations, and without limitation, the spinal fixation plate may have a thickness in a range of about 1.5 mm to about 30 mm (e.g., about 5 mm to about 25 mm, about 15 to about 20 mm, or any value or range of values therein). The spinal fixation plate may vary in cross-section thickness to provide a minimized thickness at the perimeter, while maintaining structural strength and stability with a somewhat thicker central thickness. For example, and without limitation, the perimeter thickness may be in a range of 1.5 mm to about 20 mm (e.g., about 10 mm to about 18 mm, about 12 mm to about 16 mm, or value or range of values therein), and the thickness along the central portion of the low-profile plate may be in a range of about 10 mm to about 30 mm (e.g., about 15 mm to about 25 mm, about 18 mm to about 22 mm, or any value or range of values therein). The smaller perimeter thickness may provide for easier insertion of the low-profile fixation plate into lateral incisions, posteriolateral incisions, posterior incisions, etc., allowing the avoidance of an additional incision to insert the low-profile fixation plate. In some implementations, and without limitation, the thickness may smoothly taper from the central portion of the plate to the perimeter thereof, providing a smooth transition of thickness and no hard edges.

In some implementations, and without limitation, the low profile lateral fixation plate may have a length in a range from about 25 mm to about 110 mm (e.g., about 40 mm to about 85 mm, or any value or range of values therein). The length of the fixation plate may vary depending on the particular vertebrae that are targeted for the fusion procedure (e.g., a fusion of cervical vertebrae may require a shorter fixation plate, while a fusion of thoracic or lumbar vertebrae may require a longer fixation plate). The length of the fixation plate may be made in incremental sizes (e.g., 5 mm increments) to accommodate vertebrae of different sizes along the spine.

In further aspect, the present invention relates to a method fusing adjacent vertebral bones, including making an incision in a human being to expose an intervertebral disc, implanting an intervertebral spacer between a first vertebra and an adjacent second vertebra, inserting at least one screw into each of the first and second vertebra, inserting a low-profile spinal fixation plate into the incision, and attaching the low-profile fixation plate to the screws to fix the position of the first and second vertebrae relative to each other. In some implementations, and without limitation, the screws may include a specially textured metal surface that interfaces with specially textured metal surfaces of the low-profile fixation plate. The specially textured metal surfaces of the screws and the low-profile fixation plate may include a metal material that is acceptable for human implantation and that may be capable of undergoing cold welding (e.g., titanium and titanium alloys). In some implementations, and without limitation, attaching the low-profile fixation plate to the screws may include tightening the specially textured surfaces of the low-profile fixation plate onto the specially textured surfaces of the screws with sufficient pressure to cause cold welding between the specially textured surfaces of the screws and the low-profile fixation plate.

It is therefore an object of the present invention to provide spinal fixation system for correcting a diseased or injured spine that is capable of fixing the adjacent vertebrae in a spinal fusion procedure. The fixation system of the present invention is capable of immobilizing the adjacent vertebrae for fusion, facilitating fusion of vertebrae (e.g., thoracolumbar or other vertebrae), and reducing spinal deformity, thereby allowing a patient to return to a normal quality of life.

It is a further object of the present invention to provide a surgical device system and method that reduces surgery time for spinal procedures.

It is a further object of the present invention to provide a more reliable vertebral fixation system than those that are currently available.

It is a further object of the present invention to provide a fixation system that includes a lateral fixation plate for attachment to the lateral portions of the vertebral bodies (or other portions of the vertebrae or vertebral bodies) of adjacent vertebrae, which has low profile (e.g., a reduced thickness) that allows the low-profile fixation plate to be inserted and implanted on adjacent vertebrae through the same incision used to excise a diseased intervertebral disc. This aspect of the invention is particularly useful in that it avoids the necessity of an additional incision for the implantation of the low-profile fixation plate or another fixation device, and thereby reduces the morbidity of the patient associated with the extra incision.

It is also an object of the present invention to provide a spinal fixation system that may be efficiently surgically implanted without delays due to an extra incision for the implantation of the low-profile fixation plate.

It is a further object of the present invention to provide a spinal fixation system that requires the implantation of fewer screws (e.g., two screws) into the vertebrae for attaching a fixation plate to the vertebrae.

It is a further object of the present invention to provide a spinal fixation system that includes a fixation plate and implant screws that are stably and reliably fastened together, thereby avoiding slippage or movement between the lateral fixation plate and the implant screws.

It is a further object of the present invention to provide a spinal fixation system that includes surgical implant screws and a low-profile fixation plate that fuse together when they are joined by fastening members (e.g., a threaded locking nuts). The fusion may be the result of cold welding of the interfacing surfaces of the implant screws and the low-profile fixation plate.

Additional objects of the invention will be apparent from the detailed descriptions and the drawings provided herein.

### BRIEF DESCRIPTION OF THE DRAWINGS

FIG. 1 provides an illustrated top perspective view of the lateral fixation plate of the present invention.

FIG. 2 provides an illustrated perspective view of a side lateral fixation plate of the present invention.

FIG. 3 provides an illustrated view of the bottom of the lateral fixation plate of the present invention.

FIG. 4 provides an illustrated perspective view of a screw and a fastening member of the present invention.

FIG. 5 provides an illustrated perspective view of the low-profile fixation plate spinal fixation system of the present invention, in the process of being attached to a lateral side of a vertebral column.

FIG. 6 provides an illustrated perspective view of the low-profile fixation plate spinal fixation system of the present invention, attached to a lateral side of a vertebral column.

The drawings and descriptions thereof provided in the present application are meant for illustrative purposes. The features of the drawings may not be to scale, and the drawings are not intended to limit the invention in any way. Some terms used herein to refer to the lateral fixation plate or portions thereof may be used in relation to the drawings, and should not be understood to limit the invention. For example, the words “top side” and “bottom side” may be used in the present application to describe portions of the lateral fixation plate shown in FIGS. 1 and 3, respectively. However, one of ordinary skill in the art will understand that the “top side” of the lateral fixation plate shown in FIG. 1 may be positioned laterally when the lateral fixation plate is implanted on lateral portions of vertebrae, and that the “bottom side” of the lateral fixation plate shown in FIG. 3 may be positioned medially when the lateral fixation plate is implanted on lateral portions of vertebrae.

### DETAILED DESCRIPTION

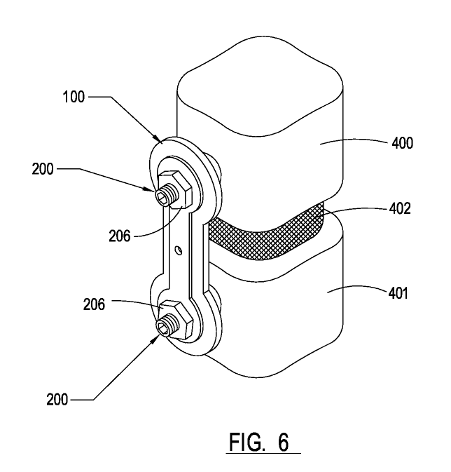

Referring to the drawings wherein like reference characters designate like or corresponding parts throughout the several views, and referring particularly to the illustrated embodiment of FIGS. 1-6, it is shown that the present invention relates to a spinal fixation system for correcting spinal compression and other spinal conditions that includes a low-profile fixation plate 100 having first and second holes 101 and 102 therein for receiving implant screws; implant screws 200 having a lower threaded portion 201, a collar or protrusion (e.g., bulb 202), and an upper threaded portion 204 configured to be inserted through one of the first holes 101 or the second hole 102; fastening members for securing the low-profile fixation plate to the implant screws 200 (e.g., a threaded nut 206); and plate holder tool 300 configured to insert the low-profile fixation plate 100 into an incision and hold the low-profile fixation plate 100 in position while it is attached to the implant screws 200. The separate parts of the system work in concert to allow fixation of two adjacent vertebrae of a patient.

By fixing the relative position of the two adjacent vertebrae, the spinal fixation system may facilitate fusion of the two adjacent vertebral bones. The low-profile fixation plate is configured to allow attachment to the lateral portions of the bodies of the two adjacent vertebrae. However, it is to be understood that the spinal fixation system of the present invention may be applied to other portions of the vertebrae (e.g., other portions of the body, lateral masses, etc.). It is to be understood that the present invention is not limited to the following detailed description, and that the scope of the present invention includes variations and modifications.

The spinal fixation system may include a low-profile plate having holes therein for connecting with surgical implant screws. The spinal fixation system may have two or more holes for receiving two or more spinal fixation screws for implantation into the vertebrae of a damaged spine (e.g., one having a diseased intervertebral disc). FIGS. 1-3 illustrate an exemplary low-profile fixation plate 100 having first and second holes 101 and 102 therein for receiving implant screws. The low-profile fixation plate may have a reduced thickness to allow it to be inserted into the same incision used to access an intervertebral disc for excision and replacement with a spacer (e.g., a lateral incision, a posteriolateral incision, a posterior incision, etc.). The thickness of the low-profile fixation plate may be in a range of from about 1.5 mm to about 30 mm (e.g., from about 5 mm to about 25 mm, from about 15 mm to about 20 mm, or any value or range of values therein). As shown in FIGS. 1-3, the thickness of the low-profile fixation plate 100 may vary from its center to its perimeter. For example, and without limitation, the perimeter thickness may be in a range of 1.5 mm to about 20 mm (e.g., about 10 mm to about 18 mm, about 12 mm to about 16 mm, or value or range of values therein), and the thickness along the central portion of the low-profile fixation plate may be in a range of about 10 mm to about 30 mm (e.g., about 15 mm to about 25 mm, about 18 mm to about 22 mm, or any value or range of values therein). The smaller perimeter thickness may provide for easier insertion of the low-profile fixation plate into lateral incisions, posteriolateral incisions, posterior incisions, etc., allowing the avoidance of an additional incision to insert the low-profile fixation plate. In other implementations, and without limitation, the thickness may smoothly taper from the central portion of the plate to the perimeter thereof, providing a smooth transition of thickness and no hard edges.

The low-profile fixation plate may also have various elongate perimeter shapes, such as an ellipse, a rounded rectangular shape, a dumbbell shape, a dog bone shape, etc. In some embodiments, and without limitation, the low-profile fixation plate may have a reduced perimeter profile to minimize its size to facilitate insertion through an incision (e.g., a lateral or posteriolateral incision for accessing an intervertebral disc), minimize displacement or damage to tissues adjacent to the targeted vertebrae, and reduce materials cost of the low-profile fixation plate, but without sacrificing the structural strength and integrity of the plate (e.g., a dumbbell or dog bone shape). As shown in FIG. 1, the exemplary low-profile fixation plate 100 may have a dumbbell shape with two screw holes 101 and 102 in the longitudinal ends of the low-profile fixation plate. The dumbbell shape of the low-profile fixation plate 100 reduces the size of the low-profile fixation plate 100 in the central area of the plate, thereby reducing its overall size. The dumbbell shape has no practical impact on the strength of the low-profile fixation plate 100 because it is manufactured from strong metal materials (e.g., titanium, surgical steel, niobium, and combinations thereof).

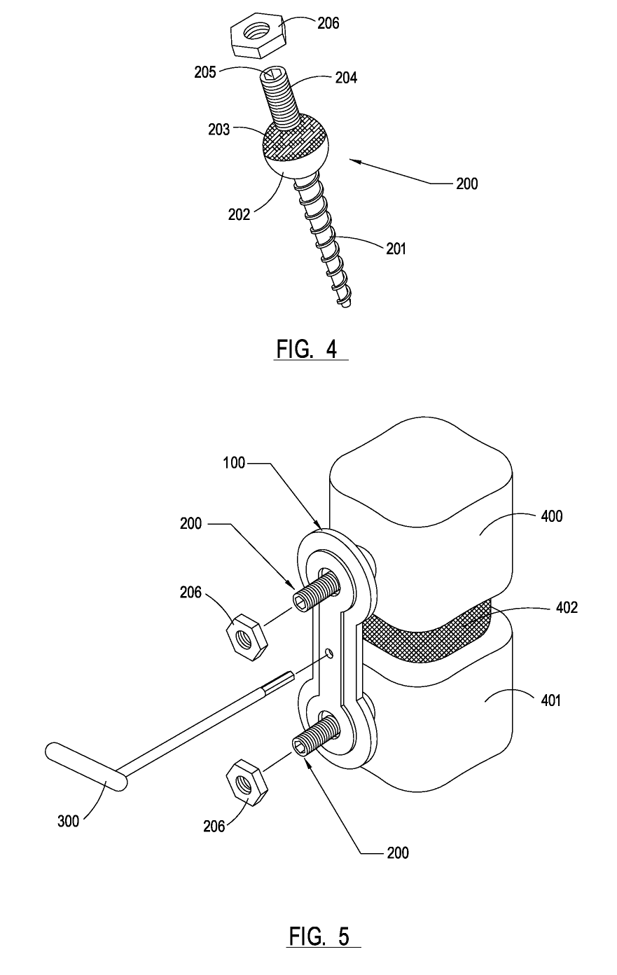

The low-profile fixation plate may include screw holes designed to receive implant screws (e.g., implant screws as shown in FIG. 4). In some implementations, and without limitation, the screw holes may have a greater diameter than the portions of the implant screw that is to be passed through the screw hole (e.g., upper threaded portions 204 of the implant screws 200 shown in FIG. 4). The increased diameter of the screw holes may allow the lateral fixation plate to accommodate the implantation of the implant screws at various angles in the vertebrae. For example, the implant screws may not always be implanted into adjacent vertebrae such that their longitudinal axes are parallel. In some implementations the implant screws may be implanted into the adjacent vertebrae at acute or obtuse angle relative to one another. For example, and without limitation, the diameter of the screw holes may be such that they can accommodate implantation angles of the implant screws of about 60° to about 90° relative to the surface of the vertebrae. Without limiting the invention, FIGS. 1-3 and 5 show lateral fixation plate 100 having screw holes 101 and 102 with a diameter larger than the upper threaded portions 204 of the implant screws 200, allowing the lateral fixation plate 100 to couple with the implant screws 200 when implanted into the vertebrae 400 and 401 at various angles.

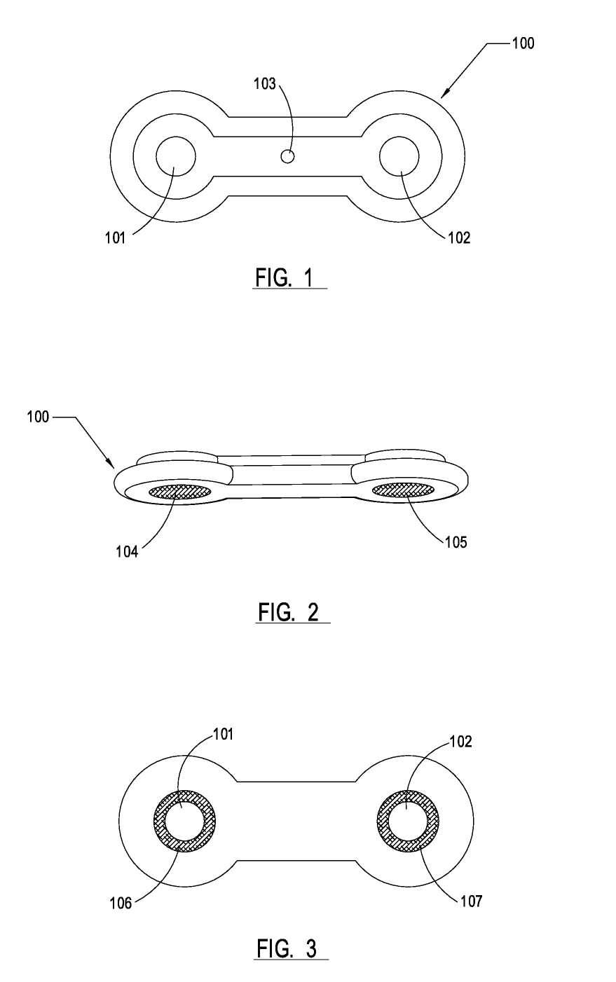

In some embodiments, and without limitation, the low-profile fixation plate may have an interfacing surface surrounding the screw holes for interfacing with a collar or protrusion of the implant screws. The interfacing surface may be flat, concave, or convex; may be a recess having one of various shapes such as spherical cap, ellipsoidal cap, a prolate spheroid cap, an oblate spheroid cap, conical, a rounded cone, cylindrical, pyramidal, star, polygonal prism (e.g., rectangular, hexagonal, etc.), or other shapes; or may be a protrusion having one of various shapes such as spherical cap, ellipsoidal cap, conical, cylindrical, pyramidal, star, polygonal prism (e.g., rectangular, hexagonal, etc.), or other shapes. Without limiting the invention, FIGS. 2-3 show an exemplary low-profile fixation plate 100 that may include concavities 104 and 105 that each surround one of the holes 101 and 102. These concavities are on the bottom side of the low-profile fixation plate 100 and are the points of interface between the low-profile fixation plate 100 and the implant screws 200.

In some implementations, and without limitation, the interfacing surfaces surrounding the screw holes may have specially textured surface for created better bite or purchase between the low-profile fixation plate and the implant screws. For example, and without limitation, the interfacing surfaces may have a high-friction surface that may include special texturing, such as a surface that has been scratched, roughened, scribed, scored, hatched, etc. by an abrasive washing or scouring process to remove contaminants and/or create the textured surface. In other examples, and without limitations, the interfacing surfaces may have a textured surface (e.g., scratched, roughened, scribed, scored, hatched, cross-hatched, stippled, etc.) created by another or additional process. The specially textured interfacing surfaces of the low-profile fixation plate may fuse with interfacing surfaces of the implant screws (which may also be specially textured) when significant pressure is applied to squeeze the interfacing surfaces together (e.g., by a tightening nut). The specially textured interfacing surfaces of the low-profile fixation plate and the implant screws may fuse together when pressed together under high pressure due to the phenomenon of cold welding, which can occur between specially textured surfaces of the same metal at high pressure. For example, and without limitation, the specially textured surfaces of the low-profile fixation plate and the implant screws may be comprise specially textured titanium or titanium alloys (e.g., alloys of Ti, Al, and/or Nb), which can undergo cold welding.

Without limiting the invention, FIGS. 2-3 show that the exemplary low-profile fixation plate 100 may have the concavities 104 and 105 may have specially textured surfaces 106 and 107. For example, and without limitation, the specially textured surfaces 106 and 107 may be scribed in various patterns (having scratches with diameters in a range of about 1 μm to about 1 mm, e.g., from about 5 μm to about 500 μm, from about 10 μm to about 250 μm, or any value or range of values therein), such as hatching, spiraling, zigzags, diamond patterns, a random pattern, and/or other patterns or combinations of patterns. The specially textured surfaces 106 and 107 may be designed to interact with specially textured surfaces 203 of implant screws 200 (see, e.g., FIG. 4). More specifically, the specially textured surfaces 106 and 107 may fuse with the specially textured surfaces 203 of the implant screws 200 when the low-profile fixation plate is tightened into position by fastening members 206 (see, e.g., FIG. 4). The specially textured surfaces 106 and 107 of the low-profile fixation plate and the specially textured surfaces 203 of the implant screws 200 may be comprise specially textured stainless steel, titanium, and/or titanium alloy (e.g., alloys of Ti, Al, and/or Nb).

The implant screws may have multiple design components, including a lower threaded portion for insertion and anchoring the screw into bone, a collar or protrusion that includes an interfacing surface for contacting and interfacing with the low-profile fixation plate, and a head that may include an upper threaded portion for receiving a fastening member (e.g., a nut) that is threaded over the upper threaded portion and over the low-profile fixation plate to hold the low-profile fixation plate in position over the implant screw. The upper threaded portion may have a recess in an end thereof for receiving a driving tool (e.g., Allen head, a torx head, star head, Phillips head, etc.), to allow the driving tool to insert the implant screw into the targeted bone. The collar or protrusion may have a portion having a convex or protruding interfacing surface having a particular shape (e.g., a spherical cap, ellipsoidal cap, a prolate spheroid cap, an oblate spheroid cap, conical, a rounded cone, cylindrical, pyramidal, star, polygonal prism (e.g., rectangular, hexagonal, etc.), or other shapes) that is complementary or at least partially complementary to a corresponding interfacing surface on the low-profile fixation plate. In other examples, and without limitation, the implant screws may have a collar or protrusion that has a concave or recessed interfacing surface (e.g., a recess having a shape such as a spherical cap, ellipsoidal cap, a prolate spheroid cap, an oblate spheroid cap, conical, a rounded cone, cylindrical, pyramidal, star, polygonal prism (e.g., rectangular, hexagonal, etc.), or other shapes) and the fixation plate may have convex or protruding complementary surface for insertion into the concave or recessed interfacing surface of the implant screw.

The lower threaded portion of the implant screws may various threading designs. In implementation, and without limitation, the implant screws may have triple lead thread to allow for quick insertion of the implant screws and efficient implantation. In other implementations, and without limitation, the lower portion may be a self-tapping screw allowing for a one-step implant process. In still other implementations, and without limitation, the lower threaded portion may have threading of a uniform circumference throughout. In further implementations, and without limitation, the lower threaded portion may have threading having a uniform circumference and a core having a varying or tapering diameter. The latter implementation may have a core having a greater diameter in the upper portion of the lower threaded portion designed for anchoring in the denser cortical bone and a smaller diameter core in the lower portion of the lower threaded portion for anchoring in the distal and less dense cancellous bone.

In some implementations, and without limitation, the implant screws may be cannulated, having a cannula running through the entire length of each of the implant screws along its longitudinal axis. The cannula may be configured to receive a surgical guide wire or pin (e.g., k wire) that may be inserted into the bone prior to the insertion of the implant screw into the incision. The guide wire and cannula may allow for accurate placement and angling of the lower threaded portion of the implant screw on the surface of the bone.

Without limiting the invention, FIG. 4 shows an exemplary implant screw 200 that may have a lower threaded portion 201 that is configured to be screwed into bone tissue (e.g., the body of a targeted vertebra), a bulb 202 (protrusion or collar) that is connected to the proximal end of the lower threaded portion, and an upper threaded portion 204 that is connected to the bulb 202 at 180° relative to the lower threaded portion and is coaxial with both the bulb 202 and the lower threaded portion 201. The lower threaded portion 201 may comprise a surgically acceptable metal, such as surgical steel, titanium, titanium alloys, etc. The bulb 202 is continuous with the lower threaded portion 201 and may comprise the same or different material (e.g., titanium, titanium alloys, surgical steel, etc.). Bulb 202 has a rounded shape (e.g., spherical, oblate, prolate, ellipsoidal, etc.), and the upper portion of the bulb 202 may be symmetrical on any plane running along the longitudinal axis of the implant screw 200. However, in other implementations, the bulb may have other shapes, such as a rounded cone shape, a double rounded cone shape, etc. In still other implementations, the bulb may be replaced by a collar or protrusion having other shapes as described herein. In any case, the concavities 104 and 105 may have a shape that is at least partially complementary to the interfacing surfaces of the implant screws to allow a close fit between the specially textured surfaces 203 and 106 and 107 to facilitate a tight fit. In some implementations, and without limitation, the complementary fit of the bulb and the interfacing surface on the low-profile fixation plate may facilitate cold welding between the interfacing surfaces.

The upper threaded portions 204 of the implant screws 200 are coaxial with both the bulb 202 and the lower threaded portions 201. The implant screws 200 may have a recess 205 in the distal end of the upper threaded portion 204 for receiving a driver head so that the implant screw may be screwed into bone tissue (e.g., a body of a vertebra). As explained above, the upper threaded portions 204 are designed to be passed through the holes 101 and 102 of the lateral fixation plate 100. This configuration allows the fastening means 206 to thread over the upper threaded portions 204 and the lateral fixation plate 100 to fix the lateral fixation plate in position on the vertebrae.

The fastening members for securing the low-profile fixation plate and the implant screws may be of various kinds of nuts that are appropriate for medical use (e.g., a hex locking nuts), and that can thread onto the upper threaded portion of the implant screws. The fastening members may be configured to tighten down the low-profile fixation plate onto the collar or protrusion of the implant screws. In some implementations, and without limitation, the collar or protrusion of the implant screw and/or the interfacing surface of the low-profile lateral fixation plate may have a specially textured surface, and the cold welding between the specially textured surfaces of the collar or protrusion and the low-profile fixation plate may occur as the fastening member is tightened over the low-profile fixation plate.

In some embodiments, and without limitation, the fastening members may be tulip head nuts that may be threaded onto the upper threaded portion of the implant screws. The tulip head nuts may be used to connect the low-profile lateral fixation plate fixation system to other fixation systems (e.g., another low-profile lateral fixation plate fixation system, or other surgical implants) on adjacent vertebrae via surgical rods threaded through the tulip heads. In such embodiments, an additional locking nut may be included in the system to thread into the tulip head nut over the surgical rod.

Without limiting the invention, FIGS. 4-5 show an exemplary locking nut 206 which may be configured to tighten down the specially textured surface 106 or 107 of the low-profile fixation plate 100 onto the specially textured surface 203 of the bulb 202 to exert substantial pressure between the specially textured surface 203 and the specially textured surface 106 or 107 of the lateral fixation plate 100, thereby facilitating cold welding between the specially textured surfaces. The locking nut 206 may be threaded and tightened onto the upper threaded portion 204 and over the low-profile fixation plate 100 by a manual or motorized driving tool.

In some embodiments, the surgical fixation system may further include a plate holder, which may also function as an anti-torque tool, for holding and stabilizing the low-profile fixation plate during an implantation procedure. In some implementations, and without limitation, the low-profile fixation plate may have a recess (e.g., near a central area of the low-profile fixation plate) for receiving a head or end of the plate holder that may have a portion having a complementary to the shape of the recess. The complementary shapes of the recess and head of the plate holder may allow the plate holder to act as an anti-torque tool while the fastening members are tightened over the implant screws and the low-profile lateral fixation plate. The process of tightening the fastening members may apply substantial torque to the low-profile lateral fixation plate and the implant screws. Thus, low-profile lateral fixation plate may be held in position during the tightening process by the plate holder or another tool. The anti-torque function of the plate holder may also prevent damage to the hardware (i.e., the low-profile lateral fixation plate, the implant screws, and the fastening members), failure of the spinal fixation system, and damage to the targeted bone tissue.

In some implementations, and without limitation, the plate holder may additionally include other features for engaging with the low-profile fixation plate, such as a clamp or clasp for grasping the low-profile fixation plate (e.g., for grasping lateral edges of the low-profile fixation plate). The plate holder may also have various shapes and configurations designed to access the low-profile fixation plate positioned within various types of incisions. For example, and without limitation, the neck of the plate holder may have certain lengths and angles therein to allow the plate holder to insert the low-profile lateral fixation plate into a posteriolateral incision and/or other approaches.

In some embodiments, the recess for engaging with of the plate holder may pass through the entire thickness of the low-profile lateral fixation plate. In such embodiments, the recess may have the additional function of receiving a temporary fixation pin (not shown) therethrough, allowing the low-profile fixation plate to be held in proper position (e.g., over adjacent vertebrae and engaged with the implant screws) before the fastening members are placed on the implant screws. The recess may have a varying shape and diameter, where the portion of the recess on the top side of the low-profile fixation plate may be complementary to the corresponding head of the plate holder and the portion of the recess on the back side of the low-profile fixation plate may be sized and shaped to accommodate the temporary fixation pin having a smaller diameter than the head of the plate holder. In other embodiments, the low-profile fixation plate may have a separate hole (not shown) located adjacent to the recess, and for receiving a temporary fixation pin.

Without limiting the invention, FIG. 5 shows an exemplary plate holder 300 having a head that engages with a centrally located recess 103 in the low-profile fixation plate 100. The plate holder 300 may have a head or portion that has a complementary shape to the recess 103, allowing it to hold the low-profile fixation plate during placement of the low-profile fixation plate 100. As mentioned above, the plate holder 300 may have an additional function as an anti-torque tool. The plate holder 300 may be attached to the lateral fixation plate during the process of placing and tightening the locking nuts 206. The plate holder 300 may prevent movement (e.g., rotational movement) of the low-profile fixation plate 100 during the process of attaching the low-profile fixation plate 100 to the implant screws 200. The process of tightening the locking nuts 206 may apply substantial torque to the lateral fixation plate 100 and the implant screws 200. In some implementations the locking nuts 206 may be tightened with sufficient torque to generate cold welding between the specially textured surface 203 of the implant screws 200 and the specially textured surfaces 106 and 107 of the lateral fixation plate 200 in a range of about 40 in.-lbs. to about 100 in.-lbs. (e.g., about 60 in.-lbs. to about 90 in.-lbs., about 75 in.-lbs. to about 85 in.-lbs., or any value or range of values therein).

In some embodiments, and without limitation, the spinal fixation system of the present invention may also include additional instruments such as a template (not shown) for use in placing the implant screws. The template may be a structure that mimics the shape, screw hole placement, and/or other characteristics of the low-profile lateral fixation plate, and provides a means for guiding the placement of holes in the vertebral bones in which the screws will be placed. The template may have holes therein that correspond to the screw holes and in the low-profile lateral fixation plate. These holes (“guide holes”) may be used to guide the formation of the holes formed in the vertebrae targeted for implantation of the low-profile lateral fixation plate. In some implementations, the template may have corresponding drill/awl guides (not shown) that can be placed in the guide holes. For example, the guide holes may be threaded so that threaded drill/awl guides can be secured in the guide holes. The drill/awl guides can then be used to stabilize and guide an awl and/or a drill during the process of forming holes for the implantation of implant screws.

In some embodiments, and without limitation, the template may mimic the contours shape of the bottom of the lateral fixation plate, allowing the medical professionals installing the spinal fixation system to determine whether there are osteophytes, protuberances, or other irregularities on the target vertebrae that would prevent the lateral fixation plate from lying flush with the surface of the vertebral bones. If such irregularities are present and detected using the template, the medical professionals can remove them.

In some embodiments, and without limitation, the spinal fixation system may also include a surgical awl (not shown) for starting and/or marking locations on the targeted vertebrae at which the holes for the implant screws will be formed. The awl may be used in combination with the template to properly place and initiate the holes. In some embodiments, the drill/awl guides may be used to stabilize the awl and direct it to the proper location for the formation of the screw holes in the targeted vertebrae for the implantation of implant screws. Primer holes (e.g., having a depth in the range of about 5 mm to about 15 mm) may be created in the cortical bone of the vertebrae using the awl. In some implementations, and without limitation, a drill may be used to finish forming the holes for implant screws, which may have a depth in a range of about 10 mm to about 30 mm (e.g., about 15 mm to about 25 mm, or any other value or range of values therein). In other implementations, and without limitation, the awl may be used to form the entirety of the holes in the targeted vertebrae for the implant screws.

In some embodiments, and without limitation, the spinal fixation system may also include a drill for forming the screw holes. For example, the system may include a manual or power drill (not shown) that can be equipped with various drill bits for forming holes in bone tissue having a depth in the range of from about 10 mm to about 30 mm (e.g., about 15 mm to about 25 mm, or any other value or range of values therein). The drill may be used in combination with the awl or instead of the awl.

In some embodiments, and without limitation, the spinal fixation system may include drivers (not shown) for both the implant screws and the fastening members. The drivers may be manual or power, and each may have a torque-limiting means. The implant screw driver may include a torque-limiting means to prevent injury to the bone (e.g., microfracture of the bone cortex), and failure of the implant screws. The fastening member driver may also include a torque-limiting means that provides a torque-limiting point that creates pressure between the fastening members and the low-profile fixation plate in a preferred range. For example, and without limitation, the fastening member driver may apply torque to the fastening members in a range of about 40 in.-lbs. to about 100 in.-lbs. (e.g., about 60 in.-lbs. to about 90 in.-lbs., about 75 in.-lbs. to about 85 in.-lbs., or any value or range of values therein), which may cause cold welding between interfacing surfaces of the implant screws and the interfacing surfaces of the low-profile lateral fixation plate.

Various other instruments, devices, and materials may be included in or used together with the spinal fixation system of the present invention, including guide pins or wires (e.g., k wires), spinal implants (e.g., intervertebral spacers), dilators, surgical channels, etc. It will be understood by those skilled in the art that the foregoing list of devices and instruments does limit the elements included in the spinal fixation system of the present invention.

The present invention also relates to a method for a spinal fixation to promote fusion of dysfunctional or diseased human vertebrae (e.g., thoracolumbar vertebrae) due to degeneration of an intervertebral disc, one or more damaged vertebral body, or other spinal conditions. Without limitation, the method may include making an incision (e.g., a lateral incision, a posteriolateral incision, a posterior incision, etc.) to expose two or more adjacent vertebral bones and the intervertebral disc(s) therebetween. Once the vertebrae are exposed, the intervertebral disc tissue and/or a diseased vertebral body may be partially or completely excised from the exposed area of the vertebral column. Subsequently, a spacer material (e.g., an interbody fusion device) may be inserted between the vertebral bodies to place the previously malpositioned vertebrae in proper spacing and relative angling, and to replace one or more intervertebral discs and/or a vertebral body.

Subsequently, the spinal fixation system of the present invention may be efficiently implanted into the patient. The template of the spinal fixation system may be inserted into the incision to guide the placement of the implant screws into the bodies of the vertebrae. The template may be placed on the lateral portions of the vertebral bodies, anterior portions of the vertebral bodies, or other portions of the vertebrae. The template according to the present invention may have two or more holes therein for establishing marks, primer holes, or holes for the implant screws. However, some embodiments may be limited to two implant screw holes. The use of only two implant screws (in contrast to four or more for conventional plate implants) allows for a more efficient procedure, taking less time in the operating room and resulting in less morbidity to the patient. A surgical awl may be used to create the marks, primer holes, or holes for the insertion of the implant screws. In some implementations, the awl may be used to form primer holes for guide pins or wires that may be inserted into the primer holes. Thereafter, the implant screws, which may be cannulated, may be guided along the guide pins or wires to their proper insertion points. In such implementations, and without limitation, the implant screws may be self-tapping, and may not require the pre-formation of a hole. The implant screws may then be drilled into the bone using a torque-limited driver that prevents the insertion of the implant screws from damaging the bone tissue of the vertebrae.

In other implementations, and without limitation, a drill guide may be attached to the template to control the placement of the awl and to prevent injury to other areas of the vertebrae and other tissues. After the awl is used to form marks, primer holes, or holes, the awl may be removed from the drill guides and a drill may subsequently be placed in the drill guides to complete the holes for the implant screws. In other implementations, and without limitation, the awl may not be used, and the drill may be used to form the entire hole structure using the template and drill guides for guidance and control. Once the holes for the implant screws are formed, the implant screws may be implanted into the screw holes using the torque-limited driver. In some implementations, and without limitation, the implant screws may be implanted into the screw holes using the template and drill guide to properly position the implant screws over the screw holes in the vertebrae for insertion.

Subsequently, the plate holder may be used to insert the low-profile lateral fixation plate into the incision. The low profile of the lateral fixation plate may allow it to be inserted through the same incision that was used to access vertebrae to excise damaged tissue and implant the spacer device. The plate holder may position the lateral fixation plate such that the interfacing surfaces (e.g., specially textured interfacing surfaces) of the plate are in contact with the collars or protrusions of the implant screws. In some implementations, and without limitation, the guide wires for the implant screws may be used to guide the placement of the lateral fixation plate, where the guide wires are threaded through the screw holes. Once the low-profile lateral fixation plate is positioned on the vertebrae, a temporary fixation pin (not shown) may be placed through a centrally located hole (for receiving a plate holder and a fixation pin) in the low-profile lateral fixation plate in order to temporarily fix the position of the low-profile lateral fixation plate until the fastening members are positioned over the low-profile lateral fixation plate on the upper threaded portions of the implant screws.

Once the low-profile lateral fixation plate is positioned over the implant screws, the fastening members may be threaded onto the upper threaded portions of the implant screws. During this process, the plate holder may remain engaged with the low-profile lateral fixation plate and function as an anti-torque tool to prevent the plate from rotating or otherwise being mispositioned as the fastening members are tightened. The fastening members may be tightened into position by driver (not shown), which may be manual or motorized. In some implementations, the driver may be torque-limited to both (1) prevent over tightening that may lead to movement of the implant screws and possible damage to the bone tissue, and (2) allow sufficient pressure to be applied to between the interfacing surfaces of the implant screws and interfacing surfaces of the low-profile lateral fixation plate to result in cold welding between the interfacing surfaces. For example, and without limitation, the driver may apply torque in a range of about 40 in.-lbs. to about 100 in.-lbs. (e.g., about 60 in.-lbs. to about 90 in.-lbs., about 75 in.-lbs. to about 85 in.-lbs., or any value or range of values therein). Once the low-profile lateral fixation plate is fixed in proper position, the spinal fixation system of the present invention holds the connected vertebrae and the spacer device in proper position, such that the vertebrae are at a proper angle relative one another and nerve compression and other symptoms associated with the former diseased vertebrae are alleviated.

In some embodiments, and without limitation, the method may further include introducing a spacer, bone graft, and/or other fusion promoting materials between the vertebrae and installing a means for holding the material in place, whereby a fusion of the vertebrae may be achieved.

It will be appreciated that systems and methods of the present invention may be used to treat vertebral instability resulting from any cause, so long as patient is sufficiently healthy to undergo implantation surgery and the patient's anatomy will allow successful implantation. The spinal fixation system of the present invention facilitates stabilization of the vertebral column through fixation, as well as fusion of diseased vertebrae.

It is to be understood that variations and modifications of the present invention may be made without departing from the scope thereof. It is also to be understood that the present invention is not to be limited by the specific embodiments, components or parts disclosed herein, nor by any of the exemplary dimensions set forth in the attached illustrations.

1. A spinal fixation system, comprising:
a. a low-profile fixation plate for attachment to vertebrae having at least a first screw hole running through said low-profile fixation plate for receiving a portion of a screw, said low-profile fixation plate having a first textured surface adjacent to said first screw hole;
b. at least a first implant screw having a first protrusion, said first protrusion having a second textured surface thereon for interfacing with said first textured surface of said low-profile fixation plate and a first head for placement through said first screw hole; and
c. at least a first fastening member for attachment to said head and for securing said low-profile fixation plate over said first protrusion of said first implant screw.

2. The spinal fixation system of claim 1, further comprising an anti-torque tool engageable with said low profile fixation plate, wherein said low profile fixation plate has a recess therein for engaging said anti-torque tool.

3. The spinal fixation system of claim 2, wherein said low-profile fixation plate has a bottom side and a top side, said first textured surface is on said bottom side and said recess is centrally located in said top side.

4. The spinal fixation system of claim 1, wherein said first textured surface has a contoured shape, and said first protrusion has a shape that is complementary to said contoured shape of said first textured surface.

5. The spinal fixation system of claim 1, wherein said low-profile fixation plate further comprises a second screw hole, said second screw hole having a third textured surface adjacent thereto.

6. The spinal fixation system of claim 5, further comprising a second implant screw having a second protrusion, said second protrusion having a fourth textured surface thereon for interfacing with said third textured surface of said low-profile fixation plate and a second head for placement through said second screw hole.

7. The spinal fixation system of claim 6, wherein said first and second implant screws each have a lower threaded shaft for implanting into bone tissue and an upper threaded portion on said first and second heads, wherein said protrusions are located between said lower threaded shaft and said upper threaded portion.

8. The spinal fixation system of claim 7, wherein said spinal fixation system further comprises a second fastening member for attachment to said second head and for securing said low-profile fixation plate over said second protrusion of said second implant screw

9. The spinal fixation system of claim 1, wherein said protrusion is a collar surrounding a portion of the shaft of the screw.

10. The spinal fixation system of claim 1, wherein said second textured surface of said first implant screw and said first textured surface of said low-profile fixation plate comprise a metal material capable of undergoing cold welding.

11. The spinal fixation system of claim 1, wherein said second textured surface of said first implant screw and said first textured surface of said low-profile fixation plate are made of the same or a different material selected from the group consisting titanium, titanium alloys (e.g., alloys of Ti, Al, and/or Nb), and stainless steel.

12. The spinal fixation system of claim 10, wherein said materials of said second textured surface of said first implant screw and said first textured surface of said low-profile fixation plate are scoured.

13. The spinal fixation system of claim 1, wherein said low-profile fixation plate has a thickness in a range of about 5 mm to about 25 mm.

14. A spinal fixation plate, comprising:
a. a low profile design in which said fixation plate has a minimized thickness;
b. a first screw hole and a second screw hole for coupling with first and second surgical implant screws;
c. a bottom side having a first interfacing surface adjacent to said first screw hole and a second interfacing surface adjacent to said second screw hole, wherein said first and second interfacing surfaces have a roughened texture for interfacing with complementary interfacing surfaces of said first and second implant screws; and
d. a recess for receiving a plate holding tool.

15. The spinal fixation plate of claim 14, wherein said spinal fixation plate is for attachment to lateral sides of adjacent vertebral bodies.

16. The spinal fixation plate of claim 14, wherein said low-profile design of said spinal fixation plate allows said spinal fixation plate to be passed and implanted through an incision for excising an intervertebral disc.

17. The spinal fixation plate of claim 14, wherein said first and second interfacing surfaces of said spinal fixation plate comprise a metal material capable of undergoing cold welding.

18. The spinal fixation plate of claim 14, wherein said spinal fixation plate has a thickness in a range of about 5 mm to about 25 mm.

19. The spinal fixation plate of claim 14, wherein said complementary interfacing surfaces of said first and second implant screws each have a roughened texture, wherein said first and second interfacing surfaces and said complementary interfacing surfaces of said first and second implant screws undergo cold welding when they are squeezed together by fastening members to a predetermined torque.

20. A method of fusing adjacent vertebral bones, comprising:
a. making an incision in a human being to expose an intervertebral disc;
b. implanting an intervertebral spacer between a first vertebra and an adjacent second vertebra;
c. inserting a low-profile fixation plate into said incision; and
d. attaching said low-profile fixation plate to a first surgical screw inserted into said first vertebra and a second surgical screw inserted into said second vertebra to fix the position of said first and second vertebrae relative to each other.

21. The method of claim 20, wherein said low profile fixation plate has a first concavity and a second concavity on a bottom side thereof, said first interfacing surrounding a first screw hole for receiving a head of said first surgical screw and the second interfacing surface surrounding a second screw hole for receiving a head of said second surgical screw.

22. The method of claim 21, wherein said first and second surgical screws each have a protrusion, said protrusion of the said first screw interfacing with said first interfacing surface and said protrusion of said second screw interfacing with said second interfacing surface.

23. The method of claim 22 wherein said first and second interfacing surfaces and said protrusions of said first and second screws each have a roughened surface.

24. The method of claim 23, wherein attaching said low-profile fixation plate to said first and second vertebrae comprises:
a. positioning said low-profile fixation plate on said first and second screws such that the roughened surface of said first interfacing surface is in contact with the roughened surface of said protrusion of said first screw, and the roughened surface of said second interfacing surface is in contact with the roughened surface of said protrusion of said second screw; and
b. tightening said low-profile fixation plate to said first and second screws using first and second fastening members that are threaded over said first and second screws, wherein said first fastening members compresses the roughened surface of said first interfacing surface to the roughened surface of said protrusion of said first screw and said second fastening member compresses the roughened surface of said second interfacing surface to the roughened surface of said protrusion of said second screw.

25. The method of claim 24, wherein the compression between said first interfacing surface and said protrusion of said first screw causes the roughened surfaces to cold weld together, and the compression between said second interfacing surface and said protrusion of said second screw causes the roughened surfaces to cold weld together.

26. The method of claim 20, further comprising engaging an anti-torque tool with said low-profile fixation plate during the step of attaching said low-profile fixation plate to said first and second surgical screws.
